(19) United States
(12) Patent Application Publication
(10) Pub. No.: US 2024/0207289 A1
(43) Pub. Date: Jun. 27, 2024

(54) METHOD TO IDENTIFY AND ALLEVIATE THE SYMPTOMS OF PARKINSONISM/PARKINSON’S DISEASE

(71) Applicant: Prepaire Labs Limited, Dublin (IE)

(72) Inventors: Carl Freer, Dublin (IE); Vicente Jorge Ribas, Barcelona (ES)

(21) Appl. No.: 18/544,874

(22) Filed: Dec. 19, 2023

Related U.S. Application Data
(60) Provisional application No. 63/433,750, filed on Dec. 19, 2022.

(51) Int. Cl. A61K 31/00; A61K 31/164

(52) U.S. Cl. CPC A61K 31/658; A61K 31/164

(57) ABSTRACT

Described herein are methods of treating symptoms of neurodegenerative disorders using palmidrol and a cannabinoid.

### CROSS-REFERENCE TO RELATED APPLICATIONS

This application claims the benefit of priority to U.S. Provisional Patent Application No. 63/433,750, filed Dec. 19, 2022, the entire contents of which are incorporated herein by reference.

### BACKGROUND

Parkinson's disease (PD) and L-DOPA-induced dyskinesia (LID) are motor disorders with significant impact on the patient's quality of life. Unfortunately, pharmacological treatments that improve these disorders without causing severe side effects are not yet available. Delay in initiating L-DOPA is no longer recommended as LID development is a function of disease duration rather than cumulative L-DOPA exposure.

### SUMMARY OF THE INVENTION

In certain aspects, provided herein are methods of treating a neurodegenerative disorder comprising:
administering a CBI agonist and a cannabinoid to a subject in need thereof.

### BRIEF DESCRIPTION OF THE DRAWINGS

FIG. 1 depicts the interaction of CBD with Cannabinoid Receptor 1.

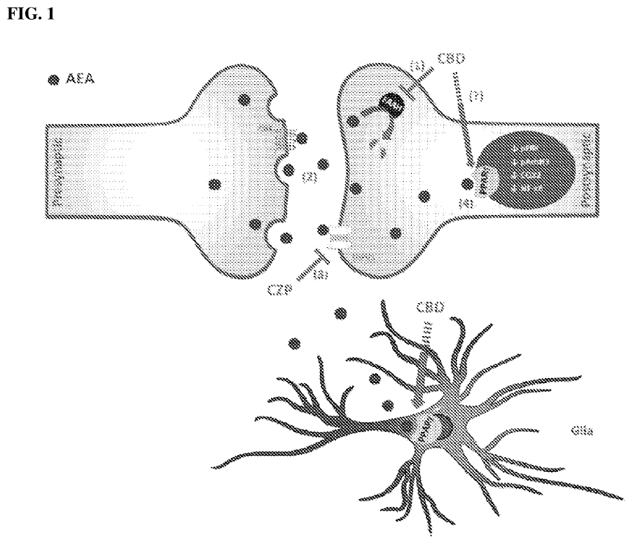

FIG. 2 shows a posterior GWAS analysis showing genes involved in Parkinson's disease with a p-value <0.05. No significant upregulated signatures related to Parkinson's disease have been identified.

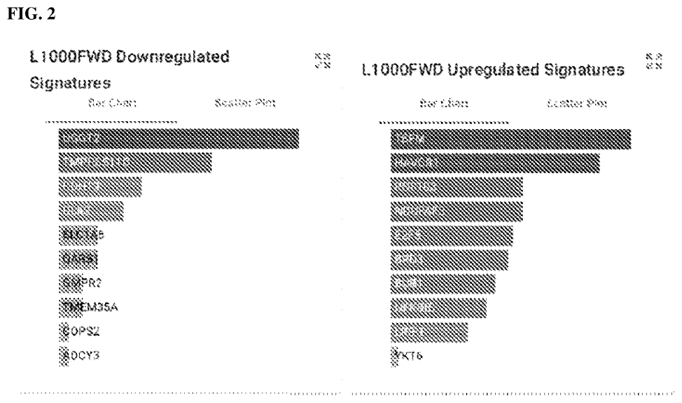

FIG. 3 shows illustrations of affinity of cannabinoids with Cannabinoid Receptor 1

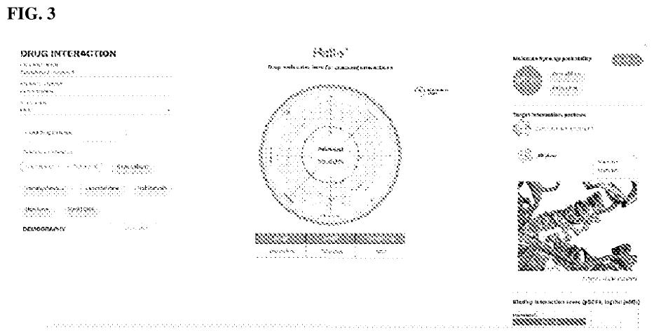

FIG. 4 shows illustrations of affinity of cannabinoids with Cannabinoid Receptor 1

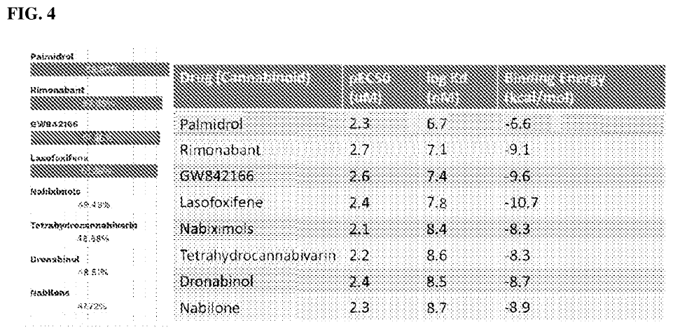

FIG. 5 shows a three-dimensional view of the affinity of dronabinol with Cannabinoid Receptor 1.

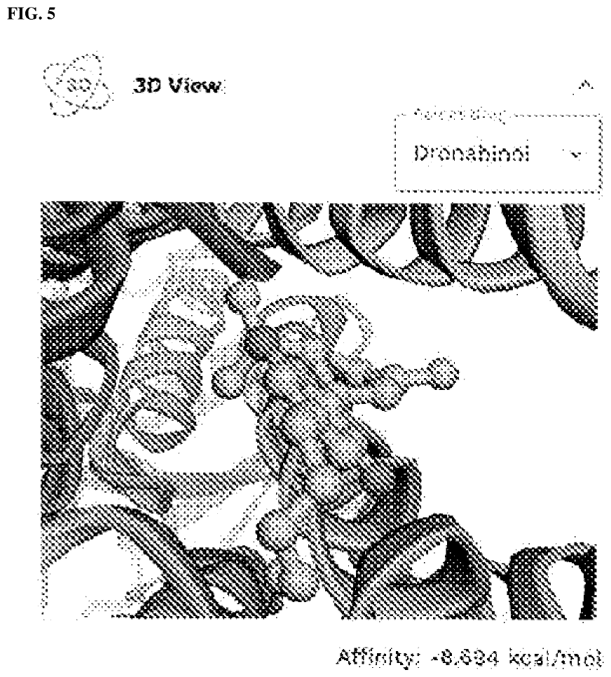

FIG. 6 shows a synergy analysis of palmidrol and dronabinol.

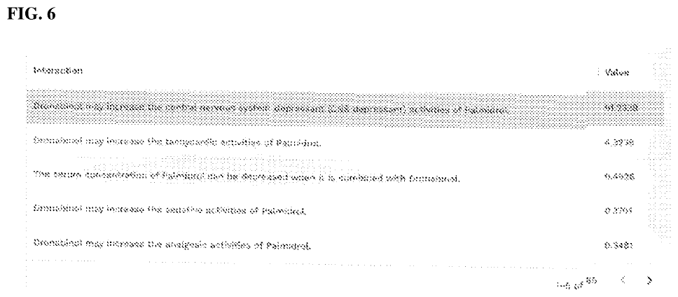

### DETAILED DESCRIPTION OF THE INVENTION

Manipulation of the endocannabinoid system could be a promising therapy to control PD and LID symptoms. In this way, phytocannabinoids and synthetic cannabinoids, such as cannabidiol (CBD), the principal non-psychotomimetic constituent of the Cannabis sativa plant, have received considerable attention in the last decade.

Cannabinoids are a class of compounds can be classified into three groups: phytocannabinoids, endocannabinoids, and synthetic cannabinoids and are generally lipophilic. Well-studied cannabinoids include tetrahydrocannabinol, cannabidiol, and cannabinol.

Clinical and preclinical evidence using the Prepaire HaiLO™ discovery tools are disclosed that suggest CBD and other cannabinoids have therapeutic effects in PD and LID.

Here, CBD pharmacology, as well as its neuroprotective effects and those of other cannabinoids are discussed. Finally, the discovery of a novel drug modulation of several pro- or anti-inflammatory factors as possible mechanisms responsible for the therapeutic/neuroprotective potential of Cannabis-derived/cannabinoid synthetic compounds in motor disorders are disclosed.

James Parkinson in “An essay on the shaking palsy” described for the first time the “paralysis agitans” or, as it is currently known, Parkinson's disease. In general, the disease is symptomatically characterized by abnormal posture, bradykinesia, resting tremor, and rigidity, among other motor and non-motor symptoms. PD is the second most frequent neurodegenerative disorder, and more than 90% of the cases are sporadic.

The pathogenesis of idiopathic PD is not fully understood. The disease is thought to be multifactorial, involving environmental and genetic factors. Studies have reported an association between PD and head injury, rural living, middle-age obesity, lack of exercise, and herbicide/insecticide exposure (paraquat, organophosphates, and rotenone), while smokers and regular drinkers of coffee have lower risk to develop PD.

The pathophysiology of PD includes—but is not restricted to—several molecular and cellular malfunctions, such as mitochondrial dysfunction, oxidative stress, misfolding and aggregation of a-synuclein, dysregulation of calcium homeostasis, and neuroinflammation. These alterations might lead to the ultimate aspect of PD, the loss of dopaminergic neurons in the nigrostriatal pathway. Therefore, more effective and tolerable treatments are necessary to retard the neurodegeneration and improve the patients' quality of life.

The studies reviewed here indicate that cannabinoids could influence the development and manifestations of PD and LID. Several mechanisms, ranging from direct changes in critical neurotransmitters such as dopamine and glutamate to indirect anti-inflammatory effects, seem to be involved. This is a “multi-targeted” compound, with an extensive range of biological effects in different neuropsychiatric disorders.

It will be apparent that multiple embodiments of this disclosure may be practiced without some or all of these specific details. In other instances, well-known process operations have not been described in detail in order not to unnecessarily obscure the present embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Palmidrol is a CB1 agonist. CB1 agonists bind to cannabinoid receptor 1 and show promise for use in treating neurological diseases, pain treatment, inflammation, and several types of cancers. CB1 agonists include rimonabant, taranabant, and methanandamide. Palmidrol is a natural fatty acid amide that is both a food component and an endogenously synthesized compound, with potential analgesic and anti-inflammatory activities. Upon administration, palmidrol may inhibit the release of pro-inflammatory mediators from activated mast cells. This may reduce inflammation and pain.

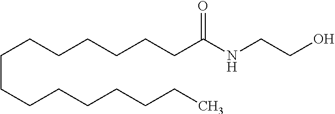 [SMILES: CCCCCCCCCCCCCCCC(=O)NCCO]

An independent drug enrichment analysis on the drug list analyzed through PREPAIRE has shown that Palmidrol is involved in the downregulation of UGGT2, TMPRSS 1 1D and ULK2. A posterior GWAS analysis has shown that these genes are involved in Parkinson's disease with a p-value <0.05. No significant upregulated signatures related to Parkinson's disease and Palmidrol have been identified in our analysis.

A synergy analysis with PREPAIRE shows that the CNS depressant properties of Palmidrol can be amplified with its combination with Dronabinol (91% probability). An exceptional high score for both efficacy and synergy-binding. This combination may be particularly relevant for alleviating the symptoms of Parkinsonism/Parkinson's disease.

Dronabinol (marketed as Marinol) is a synthetic form of delta-9-tetrahydrocannabinol (Δ⁹-THC), the primary psychoactive component of cannabis sativa. THC demonstrates its effects through weak partial agonist activity at Cannabinoid-1 (CB IR) and Cannabinoid-2 (CB2R) receptors.

A synergy analysis with PREPAIRE shows that the CNS depressant properties of Palmidol can be amplified with its combination with Dronabinol (91% probability). This combination may be particularly relevant for alleviating the symptoms of Parkinsonism/Parkinson's disease. PREPAIRE's synergy module yields a synergy score of 86 for the two molecules.

In certain aspects, provided herein are methods of treating a neurodegenerative disorder comprising:
administering a CB1 agonist and a cannabinoid to a subject in need thereof.

In certain embodiments, the CB1 agonist and cannabinoid are administered conjointly. In further embodiments, the CB1 agonist and cannabinoid are administered sequentially.

In certain embodiments, the CB1 agonist is Palmidrol. In further embodiments, the cannabinoid is Dronabinol.

In certain embodiments, the neurodegenerative disorder is selected from Parkinson's disease, Parkinsonism, and L-DOPA-induced dyskinesia. In further embodiments, the neurodegenerative disorder is Parkinson's disease. In yet further embodiments, the neurodegenerative disorder is Parkinsonism. In still further embodiments, the neurodegenerative disorder is L-DOPA-induced dyskinesia.

In certain embodiments, the subject is human. In further embodiments, administering the CB1 agonist and the cannabinoid to a subject comprises a therapeutically effective amount of the CB1 agonist and the cannabinoid.

In certain embodiments, palmidrol is administered at a dosage of up to about 1200 mg per day. In further embodiments, palmidrol is administered at a dosage of between about 300 mg to about 1200 mg per day. In yet further embodiments, palmidrol is administered at a dosage of about 300 mg per day. In yet further embodiments, palmidrol is administered at a dosage of about 400 mg per day. In still further embodiments, palmidrol is administered at a dosage of about 500 mg per day. In certain embodiments, palmidrol is administered at a dosage of about 600 mg per day. In further embodiments, palmidrol is administered at a dosage of about 700 mg per day. In yet further embodiments, palmidrol is administered at a dosage of about 800 mg per day. In still further embodiments, palmidrol is administered at a dosage of about 900 mg per day. In certain embodiments, palmidrol is administered at a dosage of about 1000 mg per day. In further embodiments, palmidrol is administered at a dosage of about 1100 mg per day. In yet further embodiments, palmidrol is administered at a dosage of about 1200 mg per day.

In certain embodiments, dronabinol is administered at a dosage of up to about 20 mg per day. In further embodiments, dronabinol is administered at a dosage of between 10 mg to about 20 mg per day. In yet further embodiments, dronabinol is administered at a dosage of about 10 mg per day. In yet further embodiments, dronabinol is administered at a dosage of about 11 mg per day. In still further embodiments, dronabinol is administered at a dosage of about 12 mg per day. In certain embodiments, dronabinol is administered at a dosage of about 13 mg per day. In further embodiments, dronabinol is administered at a dosage of about 14 mg per day. In yet further embodiments, dronabinol is administered at a dosage of about 15 mg per day. In still further embodiments, dronabinol is administered at a dosage of about 16 mg per day. In certain embodiments, dronabinol is administered at a dosage of about 17 mg per day. In further embodiments, dronabinol is administered at a dosage of about 18 mg per day. In yet further embodiments, dronabinol is administered at a dosage of about 19 mg per day. In still further embodiments, dronabinol is administered at a dosage of about 20 mg per day.

In certain embodiments, administering palmidrol and dronabinol to a subject comprises oral administration.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

As used herein, the singular forms “a,” “an,” and “the” include plural referents unless the context clearly dictates otherwise. Thus, for example, the term “module” is intended to mean one or more modules or a combination of modules. Furthermore, as used herein, the term “based on” includes based at least in part on. Thus, a feature that is described as based on some cause, can be based only on that cause, or based on that cause and on one or more other causes.

Alluding to the above, for purposes of this patent document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating a neurodegenerative disorder comprising:
administering a CB1 agonist and a cannabinoid to a subject in need thereof.

2. The method of claim 1, wherein the CB1 agonist and cannabinoid are administered conjointly.

3. The method of claim 1, wherein the CB1 agonist and cannabinoid are administered sequentially.

4. The method of claim 1, wherein the CB1 agonist is Palmidrol.

5. The method of claim 4, wherein the cannabinoid is Dronabinol.

6. The method of claim 5, wherein the neurodegenerative disorder is selected from Parkinson's disease, Parkinsonism, and L-DOPA-induced dyskinesia.

7. The method of claim 6, wherein the neurodegenerative disorder is Parkinson's disease.

8. The method of claim 6, wherein the neurodegenerative disorder is Parkinsonism.

9. The method of claim 6, wherein the neurodegenerative disorder is L-DOPA-induced dyskinesia.

10. The method of claim 5, wherein the subject is human.

11. The method of claim 1, wherein administering the CB1 agonist and the cannabinoid to a subject comprises a therapeutically effective amount of the CB1 agonist and the cannabinoid.

12. The method of claim 4, wherein palmidrol is administered at a dosage of up to about 1200 mg per day.

13. The method of claim 12, wherein palmidrol is administered at a dosage of between about 300 mg to about 1200 mg per day.

14. The method of claim 5, wherein dronabinol is administered at a dosage of up to about 20 mg per day.

15. The method of claim 14, wherein dronabinol is administered at a dosage of between 10 mg to about 20 mg per day.

16. The method of claim 11, wherein administering palmidrol and dronabinol to a subject comprises oral administration.
